(19) United States
(12) Patent Application Publication
(10) Pub. No.: US 2025/0063933 A1
(43) Pub. Date: Feb. 20, 2025

(54) APPARATUS AND METHOD FOR MANUFACTURING DISPLAY DEVICE

(71) Applicant: Samsung Display Co., Ltd., Yongin-Si (KR)

(72) Inventors: Intae Son, Yongin-si (KR); Wonseok Kim, Yongin-si (KR); Jueyoun Park, Yongin-si (KR); Kangwoo Lee, Yongin-si (KR); Dasong Lee, Yongin-si (KR); Jeongin Lee, Yongin-si (KR)

(21) Appl. No.: 18/665,576

(22) Filed: May 16, 2024

(30) Foreign Application Priority Data
Aug. 17, 2023 (KR) 10-2023-0107855

(51) Int. Cl. H10K 71/40

(52) U.S. Cl. CPC H10K 71/421

(57) ABSTRACT

An apparatus for manufacturing a display device is provided, the display device including a display panel, an adhesive layer, and a cover window, wherein the apparatus includes a gas absorption amount measuring unit that measures an amount of gas absorption of the adhesive layer at a first pressure and a first temperature, a lamination unit that laminates the adhesive layer and the cover window on the display panel, and a controller that controls the lamination unit based on information measured by the gas absorption amount measurement unit, wherein the controller controls the lamination unit to perform a lamination process only when the amount of gas absorption of the adhesive layer measured by the gas absorption amount measurement unit satisfies a first condition.

### CROSS-REFERENCE TO RELATED APPLICATION

This application is based on and claims priority under 35 U.S.C. § 119 to Korean Patent Application No. 10-2023-0107855, filed on Aug. 17, 2023, in the Korean Intellectual Property Office, the disclosure of which is incorporated by reference herein in its entirety.

### BACKGROUND

### 1. Field

One or more embodiments relate to an apparatus and method, and more particularly, to a display device manufacturing apparatus and a display device manufacturing method.

### 2. Description of the Related Art

Mobility-based electronic devices are widely used. In addition to compact electronic devices such as mobile phones, tablet personal computers (PCs) have recently been widely used as mobile electronic devices.

Such mobile electronic devices include a display device to support various functions and provide visual information such as images or videos to users. Recently, as other components for driving display devices have become smaller, the proportion of display devices in electronic devices is gradually increasing, and structures that are bendable to a predetermined angle from a flat state are also being developed.

### SUMMARY

One or more embodiments include a display device manufacturing apparatus which is capable of reducing bubbles remaining in an adhesive layer during the process of manufacturing a display device.

However, the objective is an example, and the objectives to be solved by the disclosure are not limited thereto.

Additional aspects will be set forth in part in the description which follows and, in part, will be apparent from the description, or may be learned by practice of the presented embodiments of the disclosure.

According to one or more embodiments, a manufacturing apparatus for manufacturing a display device is included, the display device including a display panel, an adhesive layer, and a cover window, is provided, the manufacturing apparatus including a gas absorption amount measuring unit that measures an amount of gas absorption of the adhesive layer at a first pressure and a first temperature, a lamination unit that laminates the adhesive layer and the cover window on the display panel, and a controller that controls the lamination unit based on information measured by the gas absorption amount measurement unit, wherein the controller controls the lamination unit to perform a lamination process only when the amount of gas absorption of the adhesive layer measured by the gas absorption amount measurement unit satisfies a first condition.

The lamination unit may include a first lamination unit that laminates the adhesive layer on the display panel, a second lamination unit that laminates the cover window on the adhesive layer, an autoclave unit that performs an autoclave process on the display device at a second pressure and a second temperature, and an ultraviolet ray treatment unit that treats the display device with ultraviolet rays.

The first temperature and the second temperature may be equal to each other.

The first pressure and the second pressure may be equal to each other.

The amount of gas absorption of the adhesive layer may include an absorption amount of N₂ of the adhesive layer.

The first pressure may be 8 bar and the first temperature may be 60 degrees Celsius. The first condition may be less than or equal to about 0.07 mg/g.

The adhesive layer may include an optically transparent adhesive.

According to one or more embodiments, a method for manufacturing a display device is included, the display device including a display panel, an adhesive layer, and a cover window, wherein the method includes measuring an amount of gas absorption of the adhesive layer at a first pressure and a first temperature, determining whether the amount of gas absorption of the adhesive layer satisfies a first condition, and laminating the adhesive layer and the cover window on the display panel when the amount of gas absorption satisfies the first condition.

The laminating may include a first lamination operation of laminating the adhesive layer on the display panel, a second lamination operation of laminating the cover window on the adhesive layer, performing an autoclave process on the display device at a second pressure and a second temperature, and treating the display device with ultraviolet rays.

The first temperature and the second temperature may be equal to each other.

The first pressure and the second pressure may be equal to each other.

The amount of gas absorption of the adhesive layer may be an absorption amount of N₂ of the adhesive layer.

The first pressure may be 8 bar and the first temperature may be 60 degrees Celsius.

The first condition may be less than or equal to about 0.07 mg/g.

The adhesive layer may include an optically transparent adhesive.

Other aspects, features and advantages in addition to those described above will become apparent from the following drawings, claims and detailed description of the disclosure.

### BRIEF DESCRIPTION OF THE DRAWINGS

The above and other aspects, features, and advantages of certain embodiments of the disclosure will be more apparent from the following description taken in conjunction with the accompanying drawings, in which:

FIGS. 1 and 2 are cross-sectional views schematically illustrating an electronic device including a display device according to an embodiment;

FIGS. 3 and 4 are plan views schematically illustrating an electronic device including a display device according to an embodiment;

FIG. 5 is a plan view schematically illustrating a display panel according to an embodiment;

FIG. 6 is an equivalent circuit diagram of a pixel according to an embodiment;

FIG. 7 is a cross-sectional view schematically illustrating a stacked structure of a display panel according to an embodiment;

FIG. 8 is a schematic block diagram of a display device manufacturing apparatus according to an embodiment;

FIGS. 9, 10, 11, 12 and 13 are schematic cross-sectional views for describing a manufacturing process of manufacturing a display device according to an embodiment;

FIGS. 14, 15 and 16 are schematic cross-sectional views for describing a manufacturing process of manufacturing a display device according to an embodiment; and

FIG. 17 is a schematic flowchart of a method for manufacturing a display device according to an embodiment.

### DETAILED DESCRIPTION

Reference will now be made in detail to embodiments, examples of which are illustrated in the accompanying drawings, wherein like reference numerals refer to like elements throughout. In this regard, the present embodiments may have different forms and should not be construed as being limited to the descriptions set forth herein. Accordingly, the embodiments are merely described below, by referring to the figures, to explain aspects of the present description. As used herein, the term “and/or” includes any and all combinations of one or more of the associated listed items. Throughout the disclosure, the expression “at least one of a, b or c” indicates only a, only b, only c, both a and b, both a and c, both b and c, all of a, b, and c, or variations thereof.

As the disclosure allows for various changes and numerous embodiments, particular embodiments will be illustrated in the drawings and described in detail in the written description. The effects and features of the disclosure, and ways to achieve them will become apparent by referring to embodiments that will be described later in detail with reference to the drawings. However, the disclosure is not limited to the following embodiments but may be embodied in various forms.

Hereinafter, embodiments of the disclosure will be described in detail with reference to the accompanying drawings, and in the description with reference to the drawings, like reference numerals refer to like elements and redundant descriptions thereof will be omitted.

It will be understood that although the terms “first,” “second,” etc. may be used herein to describe various elements, these elements should not be limited by these terms. These terms are only used to distinguish one element from another.

Singular expressions, unless defined otherwise in contexts, include plural expressions.

In the embodiments below, it will be further understood that the terms “comprise” and/or “have” used herein specify the presence of stated features or elements, but do not preclude the presence or addition of one or more other features or elements.

In the embodiments below, it will be understood when a portion such as a layer, an area, or an element is referred to as being “on” or “above” another portion, it can be directly on or above the other portion, or intervening portion may also be present.

Also, in the drawings, for convenience of description, sizes of elements may be exaggerated or contracted. For example, since sizes and thicknesses of elements in the drawings are arbitrarily illustrated for convenience of explanation, the following embodiments are not limited thereto.

In the embodiments below, an x-axis, a y-axis, and a z-axis are not limited to three axes on a rectangular coordinates system but may be construed as including these axes. For example, an x axis, a y-axis, and a z-axis may be at right angles or may also indicate different directions from one another, which are not at right angles.

When an embodiment is implementable in another manner, a predetermined process order may be different from a described one. For example, two processes that are consecutively described may be substantially simultaneously performed or may be performed in an opposite order to the described order.

In this specification, as an example of a display device according to an embodiment, an organic light-emitting display device is described, but the display device according to the disclosure is not limited thereto. As another example, the display device of the disclosure may include an inorganic light-emitting display device (Inorganic Light Emitting Display or Inorganic EL display device) or a display device such as a quantum dot light-emitting display device. For example, an emission layer included in a display element of the display device may include an organic material, an inorganic material, quantum dots, an organic material and quantum dots, or an inorganic material and quantum dots.

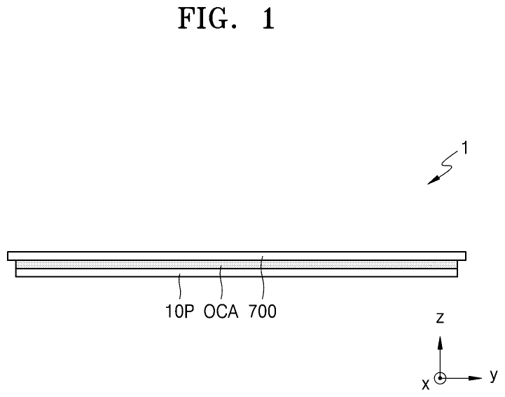

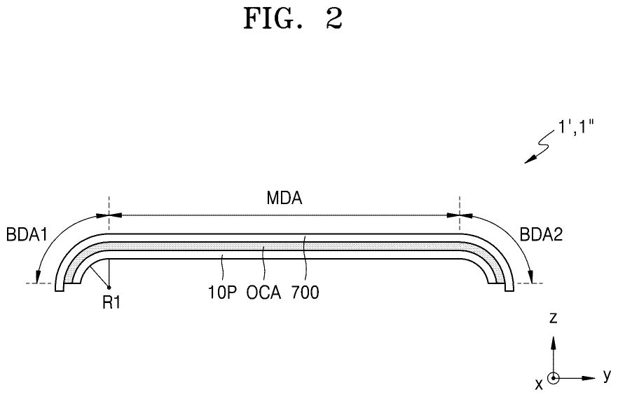

FIGS. 1 and 2 are cross-sectional views schematically illustrating an electronic device including a display device, according to an embodiment.

Referring to FIGS. 1 and 2, a display device 1, 1′, 1″ according to an embodiment may include a display panel 10P and a cover window 700 to protect an upper portion of the display panel 10P. The display device 1 may have an overall flat shape as illustrated in FIG. 1 or may have a shape in which at least some areas thereof are bent as illustrated in FIG. 2.

In the display device 1′, 1″ of FIG. 2, a display area DA may include a main display area MDA and a bendable area including a first bendable display area BDA1 and a second bendable display area BDA2. The first bendable display area BDA1 and the second bendable display area BDA2 are bendable with a radius of curvature R1. While the first bendable display area BDA1 and the second bendable display area BDA2 having the same radius of curvature R1 are illustrated in FIG. 2, in another embodiment, the first bendable display area BDA1 and the second bendable display area BDA2 may also have different curvatures of radius.

The display device 1′, 1″ may include an adhesive layer OCA formed between the display panel 10P and the cover window 700 to bond them. The adhesive layer OCA may have the same width and same area as the display panel 10P.

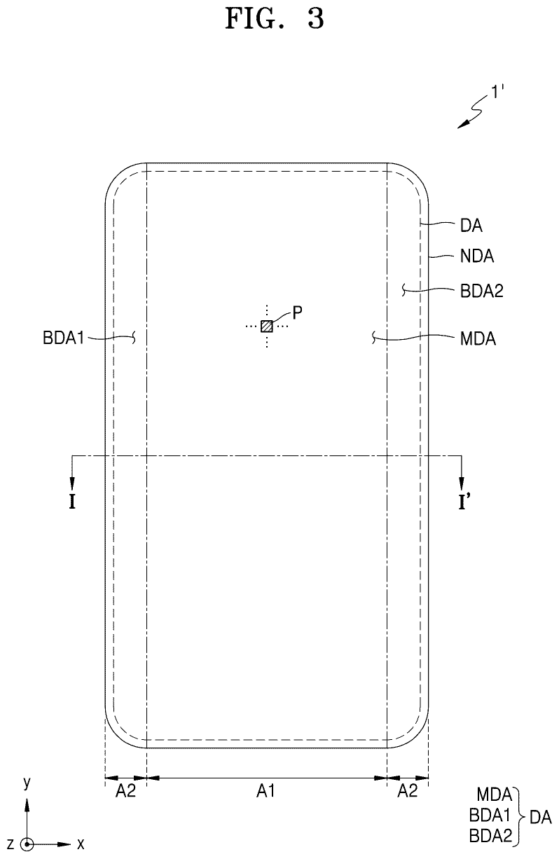

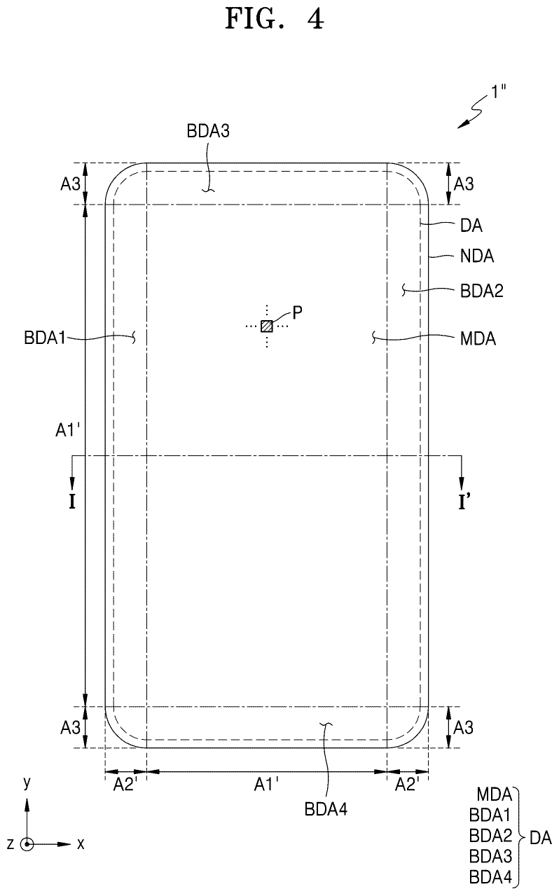

FIGS. 3 and 4 are plan views schematically illustrating an electronic device including a display device according to an embodiment.

Referring to FIGS. 3 and 4, the display device 1′, 1″ includes a display area DA and a peripheral area NDA outside the display area DA. A plurality of pixels P including a display element are arranged in the display area DA, and the display device 1′, 1″ may provide an image by using light emitted from the plurality of pixels P arranged in the display area DA. The peripheral area NDA is a non-display area in which display elements are not arranged, and the display area DA may be entirely surrounded by the peripheral area NDA.

FIGS. 3 and 4 illustrate a case where the display area DA of the display device 1′, 1″ is a rectangle with rounded corners. However, in other embodiments, the shape of the display area DA may be circular, elliptical, or a polygon such as a triangle or pentagon.

FIGS. 3 and 4 illustrate the display device 1′, 1″ in a flat form before bending, but the display device 1′, 1″ according to the present embodiment may have a three-dimensional display surface or a curved display surface as illustrated in FIG. 2. That is, FIG. 2 may correspond to a cross-section taken along a cutting line I-I′ after bending first to fourth bendable display areas BDA1 to BDA4 of the display device 1′, 1″ of FIG. 3 or 4.

When the display device 1′, 1″ includes a three-dimensional display surface, the display device 1′, 1″ may include a plurality of display areas facing different directions from each other, for example, a polygonal columnar display surface. In another embodiment, when the display device 1′, 1″ includes a curved display surface, the display device 1′, 1″ may be implemented in various forms such as a flexible, foldable, or rollable display device.

The display device 1′ of FIG. 3 may include a first area A1 and second areas A2 positioned on both sides of the first area A1. For example, the first area A1 may be a non-bendable area, and the second area A2 may be a bendable area bent with a preset curvature. The display device 1′ having a bendable area may indicate that layers constituting the display device 1′ each have a bendable area.

In an embodiment, the display area DA may include a main display area MDA corresponding to the first area A1 and a first bendable display area BDA1 and a second bendable display area BDA2 each corresponding to the second area A2.

The display device 1″ of FIG. 4 may include a first area A1′, and a second area A2′ and a third area A3 which are positioned on four edges of the first area A1′, respectively. For example, the first area A1′ may be a non-bendable area, and the second area A2′ and the third area A3 may be bendable areas that are bendable with a preset curvature. The second area A2′ is positioned on the left and right sides with the first area A1′ disposed therebetween, and may be bent based on a bending axis in a long axis direction, and the third area A3 is located above and below the first area A1′, and may be bent based on a bending axis in a short axis direction. Accordingly, a display device having a four-sided bending structure may be manufactured.

In an embodiment, the display area DA may include a main display area MDA corresponding to the first area A1′, a first bendable display area BDA1 and a second bendable display area BDA2 corresponding to the second area A2′, and a third bendable display area BDA3 and a fourth bendable display area BDA4 corresponding to the third area A3. The first bendable display area BDA1 to the fourth bendable display area BDA4 may be bent to face in different directions.

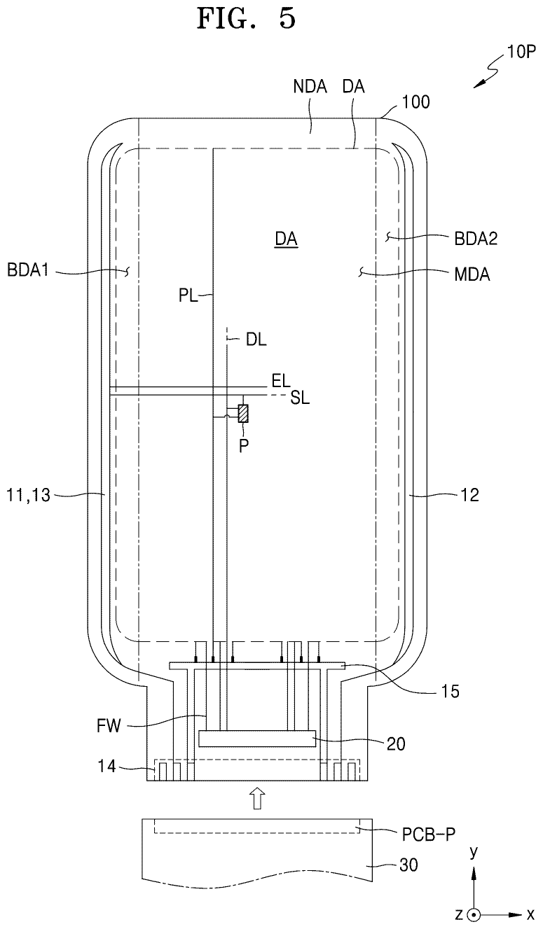

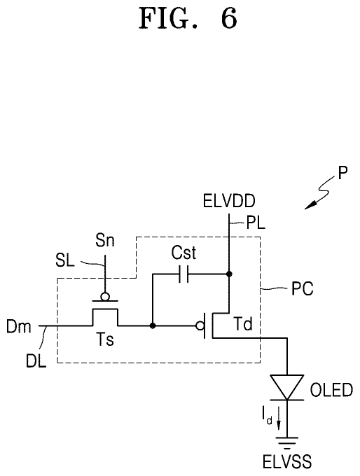

FIG. 5 is a plan view schematically illustrating a display panel according to an embodiment, and FIG. 6 is an equivalent circuit diagram of a pixel according to an embodiment.

Referring to FIG. 5, the display panel 10P may include a substrate 100, and various components constituting the display panel 10P are disposed on the substrate 100.

The display area DA may include a main display area MDA which is a non-bendable area and include the first and second bendable display areas BDA1 and BDA2 which are bendable areas adjacent to the non-bendable area. The first and second bendable display areas BDA1 and BDA2 may be arranged on both sides of the main display area MDA with the main display area MDA disposed therebetween, respectively. That is, the first and second bendable display areas BDA1 and BDA2 may be arranged adjacent to first and second scan driving circuits 11 and 12.

A plurality of pixels P may be arranged in the display area DA. Each of the plurality of pixels P may include at least one sub-pixel which includes a display element such as an organic light-emitting diode OLED. The plurality of pixels P may emit, for example, red, green, blue, or white light.

The plurality of pixels P arranged in the display area DA may be electrically connected to external circuits arranged in the peripheral area NDA, which is a non-display area. The first scan driving circuit 11, the second scan driving circuit 12, an emission control driving circuit 13, a terminal 14, and a first power supply wire 15 may be arranged in the peripheral area NDA. Although not shown, a second power supply wire may be arranged outside the first scan driving circuit 11, the second scan driving circuit 12, and the emission control driving circuit 13.

The first scan driving circuit 11 may provide a scan signal to the plurality of pixels P through scan lines SL. The second scan driving circuit 12 may be arranged in parallel with the first scan driving circuit 130 with the display area DA interposed therebetween. Some of the plurality of pixels P arranged in the display area DA may be electrically connected to the first scan driving circuit 11, and others may be connected to the second scan driving circuit 12. In another embodiment, the second scan driving circuit 12 may be omitted.

The emission control driving circuit 13 is arranged on the side in which the first scan driving circuit 11 is disposed and may provide an emission control signal to the pixels P through an emission control lines EL. FIG. 5 illustrates that the emission control driving circuit 13 is arranged only on one side of the display area DA. However, the emission control driving circuit 13, like the first and second scan driving circuits 11 and 12, may also be located on both sides of the display area DA.

The terminal 14 may be arranged in the peripheral area NDA of the substrate 100. The terminal 14 may be exposed without being covered by an insulating layer and be electrically connected to a printed circuit board PCB. A terminal PCB-P of the printed circuit board PCB may be electrically connected to the terminal 14 of the display panel 10P.

The printed circuit board PCB may be configured to transmit a signal or power from a controller (not shown) to the display panel 10. The control signal generated by the controller may be transmitted to the first scan driving circuit 11, the second scan driving circuit 12, and the emission control driving circuit 13 through the printed circuit board PCB. Additionally, the controller may provide a driving voltage ELVDD to the first power supply wire 15 and a common voltage ELVSS to the second power supply wire. The driving voltage ELVDD may be provided to the pixels P through driving voltage lines PL connected to the first power supply wire 15, and the common voltage ELVSS may be provided to opposite electrodes of pixels connected to the second power supply wire. The first power supply wire 15 may be provided below the display area DA and extend in a direction (e.g., x-direction). The second power supply wire has a loop shape with one side open, and may be arranged in the peripheral area NDA.

In addition, the controller may generate data signals, and the generated data signals may be transmitted to input lines FW through a data pad unit 20, and transmitted to the pixels P through the data lines DL connected to the input lines FW.

Referring to FIG. 6, each pixel P may include a pixel circuit PC connected to the scan line SL and the data line DL and an organic light-emitting diode OLED connected to the pixel circuit PC.

The pixel circuit PC may include a driving thin film transistor Td, a switching thin film transistor Ts, and a storage capacitor Cst. The switching thin-film transistor Ts is connected to the scan line SL and the data line DL, and may be configured to transmit, to the driving thin-film transistor Td, a data signal Dm that is input through the data line DL in response to a scan signal Sn input through the scan line SL.

The storage capacitor Cst is connected between the switching thin-film transistor Ts and the driving voltage line PL, and stores a voltage corresponding to a difference between a voltage received from the switching thin-film transistor Ts and the first power voltage ELVDD supplied to the driving voltage line PL.

The driving thin-film transistor Td is connected between the driving voltage line PL and the organic light-emitting diode OLED, and may control, in response to a voltage value stored in the storage capacitor Cst, a driving current flowing from the driving voltage line PL to the organic light-emitting diode OLED. The organic light-emitting diode OLED may emit light having a certain luminance according to a driving current Id.

While the pixel circuit PC including two thin-film transistors and one storage capacitor is described with reference to FIG. 6, the disclosure is not limited thereto. In another embodiment, the pixel circuit PC may include, for example, seven thin-film transistors and one storage capacitor. In another embodiment, the pixel circuit PC may include two or more storage capacitors.

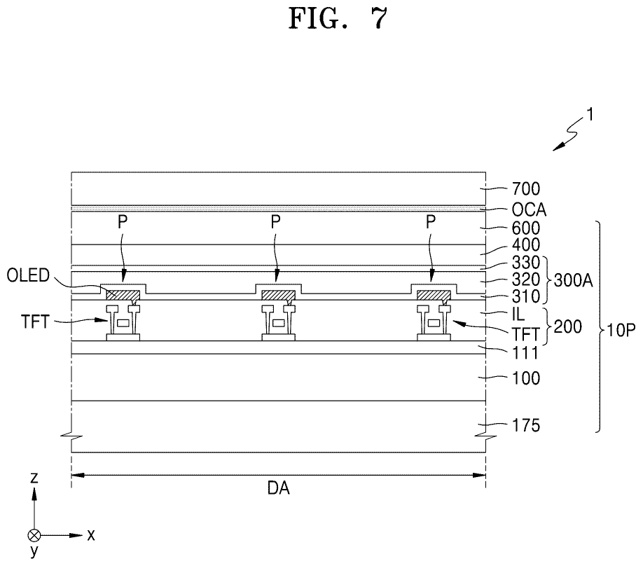

FIG. 7 is a cross-sectional view schematically illustrating a stacked structure of a display panel according to an embodiment.

Referring to FIG. 7, the display panel 10P may include a plurality of display elements for displaying an image.

Referring to FIG. 7, the display panel 10P may include a substrate 100, a display layer 200 disposed on the substrate 100, a thin film encapsulation layer 300A disposed on the display layer 200, an input sensing layer 400, an anti-reflection layer 600, and the cover window 700.

The substrate 100 may include glass or polymer resin. For example, the substrate 100 may include a polymer resin such as polyethersulfone, polyacrylate, polyether imide, polyethylene naphthalate, polyethylene terephthalate, polyphenylene sulfide, polyarylate, polyimide, polycarbonate, or cellulose acetate propionate. The substrate 100 containing polymer resin may be flexible, rollable, or bendable. The substrate 100 may have a multilayer structure including a layer containing the polymer resin described above and an inorganic layer (not shown).

A buffer layer 111 may be disposed on the substrate 100. The buffer layer 111 may reduce or block penetration of foreign substances, moisture, or external air from below the substrate 100 and may provide a flat surface on the substrate 100. The buffer layer 111 may include an inorganic insulating material such as silicon oxide, silicon oxynitride, or silicon nitride, and may have a single-layer or multi-layer structure including the materials described above.

The display layer 200 may be disposed on a front surface of the substrate 100, and a lower protective film 175 may be disposed on a rear surface of the substrate 100. The lower protective film 175 may have a function of supporting and protecting the substrate 100. The lower protective film 175 may include an organic insulating material such as polyethylene terephthalate (PET) or polyimide (PI). The lower protective film 175 may be attached to the rear surface of the substrate 100. An adhesive layer may be located between the lower protective film 175 and the substrate 100. Alternatively, the lower protective film 175 may be formed directly on the rear surface of the substrate 100, and in this case, no adhesive layer may be between the lower protective film 175 and the substrate 100.

The display layer 200 may include a plurality of pixels. The display layer 200 may include a display element layer including an organic light-emitting diode OLED as a display element, a circuit layer including a thin-film transistor TFT electrically connected to the organic light-emitting diode OLED, and insulating layers IL. The organic light-emitting diode OLED may be electrically connected to the thin-film transistor TFT to form a pixel P.

The display layer 200 may be sealed with an encapsulation member. In an embodiment, the encapsulation member may include a thin film encapsulation layer 300A as illustrated in FIG. 5. The thin film encapsulation layer 300A may include at least one inorganic encapsulation layer and at least one organic encapsulation layer. In an embodiment, the thin film encapsulation layer 300A may include first and second inorganic encapsulation layers 310 and 330 and an organic encapsulation layer 320 disposed therebetween.

In another embodiment, the encapsulation member may include an encapsulation substrate. The encapsulation substrate may be arranged to face the substrate 100 with the display layer 200 disposed therebetween. There may be a gap between the encapsulation substrate and the display layer 200. The encapsulation substrate may include glass. A sealant may be arranged between the substrate 100 and the encapsulation substrate, and the sealant may be disposed in the peripheral area NDA previously described with reference to FIG. 3 or 4. The sealant arranged in the peripheral area NDA may surround the display area DA and prevent moisture from penetrating through the sides thereof.

The input sensing layer 400 may obtain coordinate information according to an external input, for example, a touch event of an object such as a finger or a stylus pen. The input sensing layer 400 may include a touch electrode and trace lines connected to the touch electrode. The input sensing layer 400 may detect an external input by using a mutual cap method or a self-cap method.

The input sensing layer 400 may be formed on the encapsulation member. Alternatively, the input sensing layer 400 may be formed separately and then bonded to the encapsulation member through an adhesive layer such as an optically transparent adhesive. As an embodiment, the input sensing layer 400 may be formed directly on the thin film encapsulation layer 300A or the encapsulation substrate. In this case, the adhesive layer may not be arranged between the input sensing layer 400 and the thin film encapsulating layer 300A or the encapsulation substrate.

The anti-reflection layer 600 may reduce reflectance of light (external light) incident from the outside toward the display panel 10P.

In an embodiment, the anti-reflection layer 600 may include an optical plate including a phase retarder and/or a polarizer. The phase retarder may be a film type or a liquid crystal coating type, and may include a λ/2 phase retarder and/or a λ/4 phase retarder. The polarizer may also be a film type or a liquid crystal coating type. The film-type polarizer may include a stretched type synthetic resin film, and the liquid crystal coating-type polarizer may include liquid crystals arranged in a predetermined arrangement.

In an embodiment, the anti-reflection layer 600 may include a filter plate including a black matrix and color filters. The filter plate may include color filters, a black matrix, and an overcoat layer arranged for each pixel.

In an embodiment, the anti-reflection layer 600 may include a destructive interference structure. The destructive interference structure may include a first reflective layer and a second reflective layer disposed on different layers. First reflected light and second reflected light reflected from the first reflective layer and the second reflective layer, respectively, may undergo destructive interference, and thus the external light reflectance may be reduced.

The cover window 700 may be disposed on the display panel 10P. The cover window 700 may be a flexible window. The cover window 700 may protect the display panel 10P by easily bending in response to an external force without causing cracks or the like. The cover window 700 may include glass, sapphire, or plastic. The cover window 700 may be, for example, ultra-thin glass (UTG) or transparent polyimide (colorless polyimide (CPI)). In an embodiment, the cover window 700 may have a structure in which a flexible polymer layer is disposed on one side of a glass substrate, or may include only a polymer layer.

The cover window 700 is disposed on the anti-reflection layer 600 of the display panel 10P, and may be coupled to the anti-reflection layer 600 through an adhesive layer OCA such as an optically clear adhesive.

In an embodiment, the cover window 700 in FIG. 7 is illustrated as being disposed on the anti-reflection layer 600, but in another embodiment, the positions of the anti-reflection layer 600 and the input sensing layer 400 may be changed, and in this case, the cover window 700 may be coupled to the input sensing layer 400 through an adhesive layer OCA.

In an embodiment, the thickness of the adhesive layer OCA may be about 50 μm to about 300 μm. Additionally, in an embodiment, the curing rate of the adhesive layer OCA may be about 95% or more.

The adhesive layer OCA according to an embodiment may include an adhesive composition including (meth)acrylate having an alicyclic group, low-temperature glass transition (meth)acrylate, crosslinkable (meth)acrylate, and a heat curing agent including isocyanate-based compound.

In an embodiment, when the adhesive composition is analyzed using nuclear magnetic resonance (NMR) spectroscopy, the content of the heat curing agent may be about 55% or more. Nuclear magnetic resonance spectroscopy is a method of measuring a sample to be analyzed using radio frequency (RF) resonance which causes rotational transitions of atomic nuclei.

In detail, the adhesive composition may include about 5 wt % to about 15 wt % of (meth)acrylate having an alicyclic group, about 15 wt % to about 25 wt % of low-temperature glass transition (meth)acrylate, about 5 wt % to about 15 wt % of crosslinkable (meth)acrylate, and about 55 wt % to about 65 wt % of a heat curing agent including isocyanate-based compound.

The (meth)acrylate having an alicyclic group may include, for example, one or more of isobornyl (meth)acrylate, bornyl (meth)acrylate, and cyclohexyl (meth)acrylate. For example, the (meth)acrylate having an alicyclic group may be isobornyl (meth)acrylate (IBOA). Preferably, the (meth)acrylate having an alicyclic group may be (meth)acrylate whose homopolymer has a glass transition temperature of 90° C. or higher, for example, about 90° C. to about 120° C. The (meth)acrylate having an alicyclic group may be included in an amount of about 5 wt % to about 15 wt % in the adhesive composition. Within the above range, the adhesive composition may achieve the effect of increasing the peel strength and increasing modulus of the adhesive layer OCA and ensure stable folding properties of the adhesive layer OCA.

The low-temperature glass transition (meth)acrylate may include one or more of, for example, benzyl (meth)acrylate, n-butyl (meth)acrylate, sec-butyl (meth)acrylate, cyclohexyl (meth)acrylate, iso-decyl (meth)acrylate. Acrylate, n-decyl (meth)acrylate, lauryl (meth)acrylate (meth)acrylate, 2-ethylhexyl (meth)acrylate, 2-(2-ethoxyethoxy)ethyl acrylate, n-hexyl acrylate, and n-octyl (meth)acrylate. For example, the low-temperature glass transition (meth)acrylate may be 2-ethylhexyl (meth)acrylate (2-EHA). The low-temperature glass transition (meth)acrylate may be a (meth)acrylate whose glass transition temperature of homopolymer is in the range of about-100° C. to about 40° C., or in the range of about −80° C. to about 30° C., or in the range of about-75° C. to about 20° C. The low-temperature glass transition (meth)acrylate may be included in the adhesive composition in an amount of about 15 wt % to about 25 wt %.

The crosslinkable (meth)acrylate may be, for example, a (meth)acrylate having an alkyl group. The alkyl group of the (meth)acrylate having the alkyl group may be a linear or branched C1-C14 alkyl group, specifically, a C1-C8 alkyl group. The (meth)acrylate having the alkyl group may include one selected from the group consisting of methyl (meth)acrylate, ethyl (meth)acrylate, n-propyl (meth)acrylate, isopropyl (meth)acrylate, and n-butyl (meth)acrylate, t-butyl (meth)acrylate, sec-butyl (meth)acrylate, pentyl (meth)acrylate, 2-ethylhexyl (meth)acrylate, 2-ethylbutyl (meth)acrylate, octyl (meth)acrylate, isooctyl (meth)acrylate, isobornyl (meth)acrylate, isononyl (meth)acrylate, and combinations thereof. As an example, the crosslinkable (meth)acrylate may be octyl (meth)acrylate (OMA). The adhesive composition may be adjusted to have appropriate adhesive properties by using (meth)acrylate having an alkyl group with a carbon number in the above range. The crosslinkable (meth)acrylate may be included in an amount of about 5 wt % to about 15 wt % in the adhesive composition.

The heat curing agent may include an isocyanate-based curing agent. The isocyanate-based curing agent may be, for example, one selected from the group consisting of toluene diisocyanate, diphenylmethane diisocyanate, xylene diisocyanate, methylene diphenylmethane diisocyanate, isophorone diisocyanate (IPDI), cyclohexane diisocyanate, hexamethylene diisocyanate, and combinations thereof. As an example, the heat curing agent may be isophorone diisocyanate (IPDI). The heat curing agent may be included in an amount of about 55 wt % to about 65 wt % in the adhesive composition. The heat curing agent may increase the degree of cross-linking of the adhesive composition according to an embodiment, and may easily control the initial adhesive force and peel force to a required level.

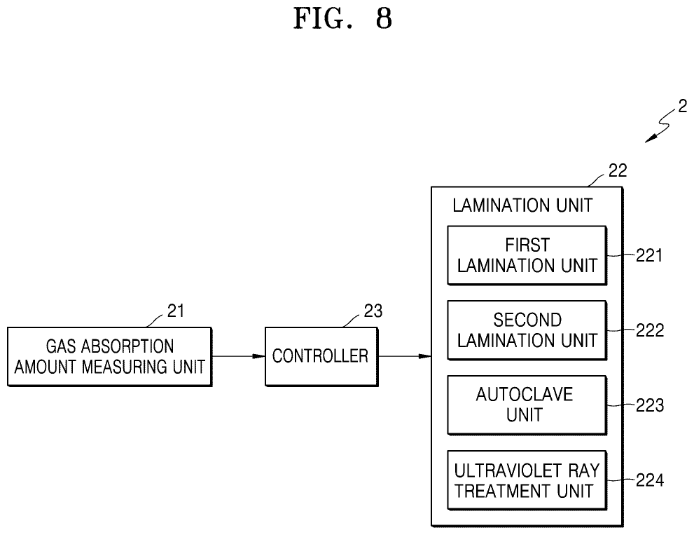

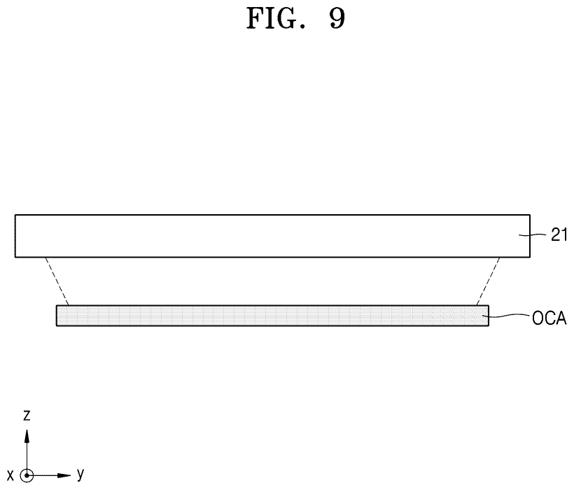

FIG. 8 is a schematic block diagram of a manufacturing apparatus for a display device according to an embodiment, and FIGS. 9 to 13 are schematic cross-sectional views for describing a manufacturing process of manufacturing a display device, according to an embodiment.

Referring to FIGS. 8 to 13, a display device manufacturing apparatus 2 may include a gas absorption amount measuring unit 21, a lamination unit 22, and a controller 23.

First, referring to FIGS. 8 and 9, the gas absorption amount measuring unit 21 may measure an amount of gas absorption of the adhesive layer OCA at a first pressure and a first temperature. For example, the IsoSORP equipment manufactured by TA Instruments may be used as the gas absorption measurement unit 21.

For example, the amount of gas absorption of the adhesive layer OCA measured by the gas absorption amount measuring unit 21 may be a N₂ absorption amount of the adhesive layer OCA. Additionally, the first pressure may be 8 bar, and the first temperature may be 60 degrees Celsius.

The controller 23 may determine whether the amount of gas absorption of the adhesive layer OCA measured by the gas absorption amount measurement unit 21 satisfies the first condition. Here, the amount of gas absorption may be expressed as the weight of the absorbed gas compared to the weight of the adhesive layer OCA. For example, the first condition may be less than or equal to about 0.07 mg/g. That is, the controller 23 may determine whether the amount of N₂ absorption of the adhesive layer OCA measured by the gas absorption amount measuring unit 21 is less than or equal to about 0.07 mg/g under the conditions of 8 bar and 60 degrees Celsius.

Referring to FIGS. 8 and 10 to 13, the lamination unit 22 may sequentially laminate the adhesive layer OCA and the cover window 700 on the display panel 10P. The lamination unit 22 may include a first lamination unit 221, a second lamination unit 222, an autoclave unit 223, and an ultraviolet ray treatment unit 224.

The controller 23 may control the lamination unit 22 based on information measured by the gas absorption amount measurement unit 21. In detail, the controller 23 may control the lamination unit 22 to perform a lamination process only when the amount of gas absorption of the adhesive layer OCA measured by the gas absorption amount measurement unit 21 satisfies the first condition. Accordingly, if the amount of gas absorption of the adhesive layer OCA satisfies the first condition, the lamination process of the adhesive layer OCA and the cover window 700 by the lamination unit 22 on the display panel 10P may be performed. Additionally, if the amount of gas absorption of the adhesive layer OCA does not meet the first condition, the lamination process of the lamination unit 22 may not be performed.

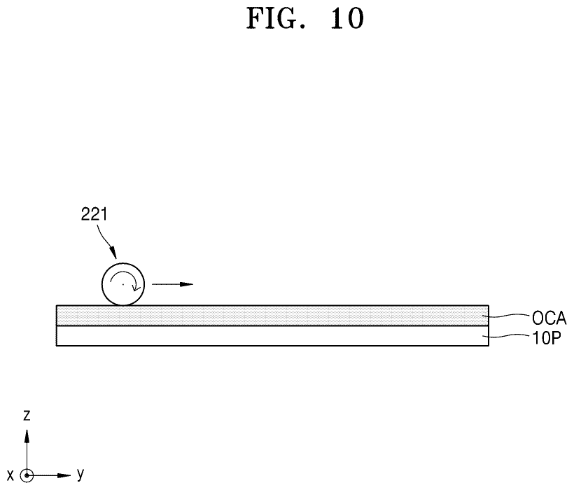

Referring to FIGS. 8 and 10, the first lamination unit 221 may laminate the adhesive layer OCA on the display panel 10P.

First, a release film (not shown) disposed on one side of the adhesive layer OCA may be removed, and then the one side of the adhesive layer OCA may be attached to the display panel 10P. The first lamination unit 221 may laminate the adhesive layer OCA on the display panel 10P through a roll lamination process. That is, the adhesive layer OCA may be attached to the display panel 10P by rotating a roller while pressing the adhesive layer OCA on the display panel 10P with the roller.

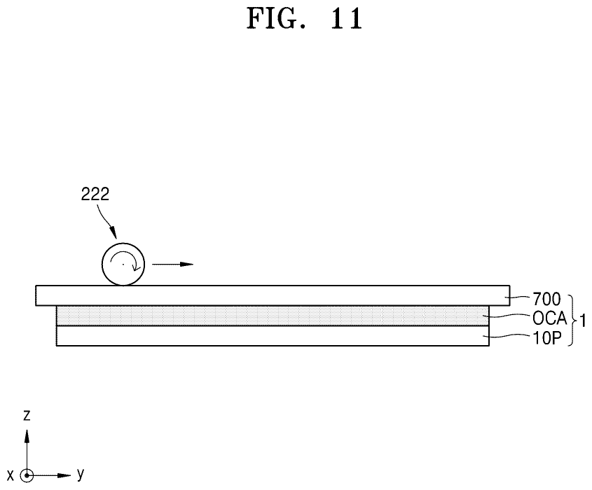

Referring to FIGS. 8 and 11, the second lamination unit 222 may laminate the cover window 700 on the adhesive layer OCA.

First, a release film (not shown) disposed on the other side of the adhesive layer OCA may be removed, and then the cover window 700 may be attached to the other side of the adhesive layer OCA. The second lamination unit 222 may laminate the cover window 700 on the adhesive layer OCA through a roll lamination process. That is, the cover window 700 may be attached to the adhesive layer OCA by rotating a roller while pressing the cover window 700 to the adhesive layer OCA with the roller.

The roll lamination process described with reference to FIGS. 10 and 11 is only an example, and the lamination process is not limited thereto. For example, the lamination process may be performed by a vacuum lamination process that vacuum-treats the display panel 10P, the adhesive layer OCA, and the cover window 700, or both a roll lamination process and a vacuum lamination process may be performed.

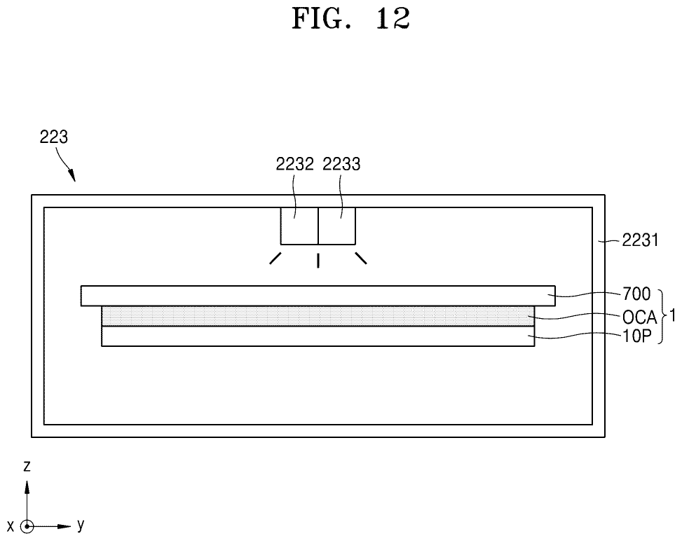

Referring to FIGS. 8 and 12, the autoclave unit 223 may perform an autoclave process on a display device 1 at a second pressure and a second temperature. The autoclave process may be a process of pressurizing the display device 1 at high temperature to remove air bubbles formed between the display panel 10P and the adhesive layer OCA, and between the adhesive layer OCA and the cover window 700. The autoclave unit 223 may include a chamber 2231, a pressure control portion 2232, and a temperature control portion 2233.

The chamber 2231 may provide an internal space where the display device 1 is placed. A passage (not shown) may be disposed at one side of the chamber 2231 to allow the display device 1 to be brought in and out. When the display device 1 is brought into the chamber 2231, the passage (not shown) disposed at the chamber 2231 may be sealed.

The pressure control portion 2232 may control the pressure of the internal space of the chamber 2231. For example, the pressure control portion 2232 may introduce external gas into the internal space of the chamber 2231 to increase the pressure inside the chamber 2231. Additionally, the pressure control portion 2232 may discharge gas from the internal space of the chamber 2231 to the outside, thereby lowering the pressure inside the chamber 2231.

The temperature control portion 2233 may control the temperature of the internal space of the chamber 2231. For example, the temperature control portion 2233 may increase the temperature inside the chamber 2231 or reduce the temperature inside the chamber 2231.

When the display device 1 is brought into the chamber 2231, the passage (not shown) disposed at the chamber 2231 is sealed, and the pressure control portion 2232 may increase the pressure inside the chamber 2231 up to the second pressure, and the temperature control portion 2233 may increase the temperature inside the chamber 2231 to the second temperature. After a specified time has elapsed at the second temperature and the second pressure, the pressure control portion 2232 may lower the pressure inside the chamber 2231 to the initial pressure, and the temperature control portion 2233 may lower the temperature inside the chamber 2231 to the initial temperature. The initial pressure may be normal pressure, and the initial temperature may be room temperature.

Here, the second temperature may be equal to the first temperature, and the second pressure may be equal to the first pressure. For example, the first temperature and the second temperature may each be 60 degrees Celsius, and the first pressure and the second pressure may each be 8 bar.

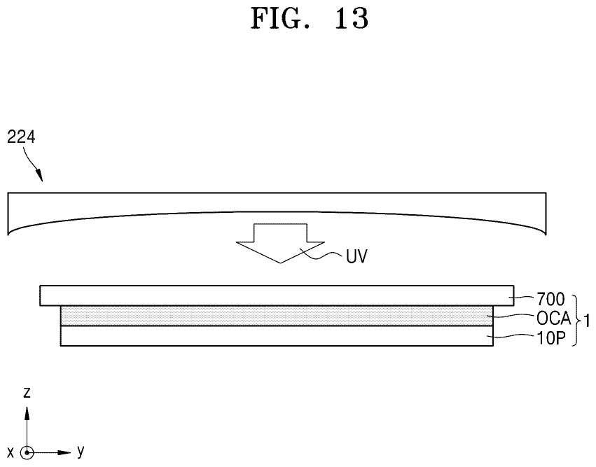

Referring to FIGS. 8 and 13, the ultraviolet ray treatment unit 224 may treat the display device 1 with ultraviolet rays. In the ultraviolet treatment process, the adhesive layer OCA may be irradiated with ultraviolet rays. As ultraviolet rays are irradiated to the adhesive layer OCA, the adhesive layer OCA is hardened so that the display panel 10P and the cover window 700 may be uniformly bonded to each other.

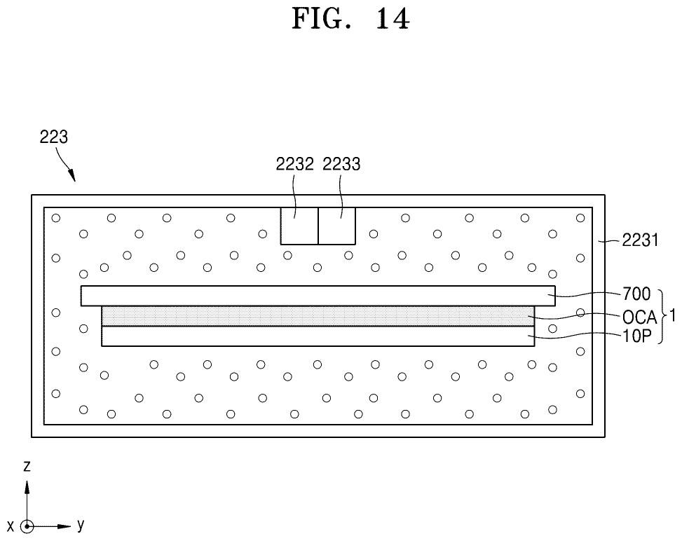

FIGS. 14 to 16 are schematic cross-sectional views for describing a manufacturing process of manufacturing a display device according to an embodiment.

Referring to FIGS. 8 and 14 to 16, the autoclave process of the autoclave unit 223 for the display device 1 is illustrated.

First, referring to FIGS. 8 and 14, the display device 1 may be brought into the chamber 2231. Here, the display device 1 may include the display panel 10P, the adhesive layer OCA, and the cover window 700. Here, the pressure control portion 2232 and the temperature control portion 2233 may not yet operate. That is, the inside of the chamber 2231 may be at room temperature and normal pressure. Air bubbles may still remain between the display panel 10P and the adhesive layer OCA, and between the adhesive layer OCA and the cover window 700. The remaining air bubbles may be visible from outside the display device 1.

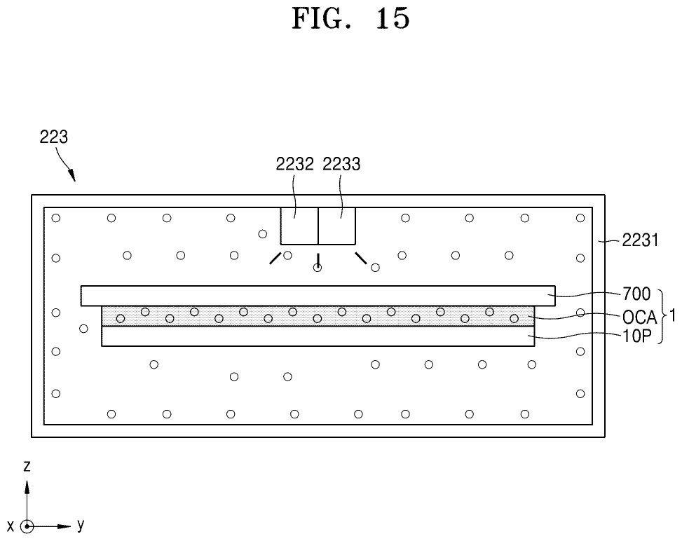

Referring to FIGS. 8 and 15, the pressure control portion 2232 and the temperature control portion 2233 may operate. The pressure control portion 2232 may increase the pressure so that the first pressure is formed inside the chamber 2231, and the temperature control portion 2233 may increase the temperature so that the first temperature is formed inside the chamber 2231. That is, the autoclave unit 223 may provide a high temperature and high-pressure environment to the display device 1.

In this process, air bubbles positioned between the display panel 10P and the adhesive layer OCA and between the adhesive layer OCA and the cover window 700 may be removed by the high temperature and high-pressure environment. However, due to the high temperature and high-pressure environment, the gas inside the chamber 2231 may be dissolved in the adhesive layer OCA. This high-temperature and high-pressure environment may last for a specified period of time.

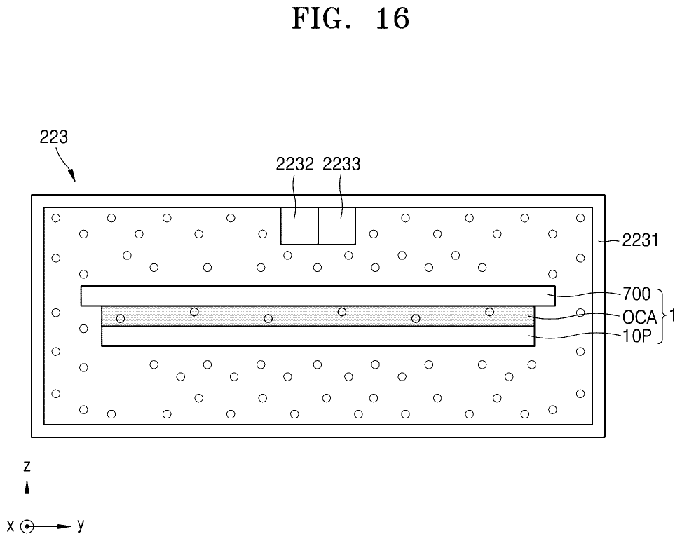

Referring to FIGS. 8 and 16, after a specified time has elapsed, the pressure control portion 2232 may lower the pressure so that normal pressure is formed inside the chamber 2231, and the temperature control portion 2233 may lower the temperature so that the room temperature is formed inside the chamber 2231. That is, the autoclave unit 223 may provide an environment of room temperature and normal pressure to the display device 1.

As the pressure and temperature inside the chamber 2231 decrease, some of the gas dissolved in the adhesive layer OCA may diffuse to the outside of the adhesive layer OCA. However, in this process, the remaining portion of the gas dissolved in the adhesive layer OCA may not diffuse to the outside of the adhesive layer OCA and may remain inside the adhesive layer OCA. The faster the pressure and temperature inside the chamber 2231 are lowered, the greater the amount of gas left inside the adhesive layer OCA. The gas remaining inside the adhesive layer OCA may be visible from the outside of the display device 1. Accordingly, the quality of the display device 1 may deteriorate.

According to the operations described with reference to FIGS. 8 and 13, the controller 23 may control the lamination unit 22 to perform a lamination process only when the amount of gas absorption of the adhesive layer OCA measured by the gas absorption amount measuring unit 21 satisfies the first condition. That is, the controller 23 may perform a lamination process only on the adhesive layer OCA which includes a relatively small gas absorption amount.

Therefore, in the operation described with reference to FIG. 15, the amount of gas absorption of the adhesive layer OCA may be relatively small in an autoclave process. Accordingly, in the operation described with reference to FIG. 16, the amount of gas remaining inside the adhesive layer OCA may be relatively small. As a result, the amount of bubbles visible from the outside of the display device 1 may be reduced, and the quality of the display device 1 may be improved.

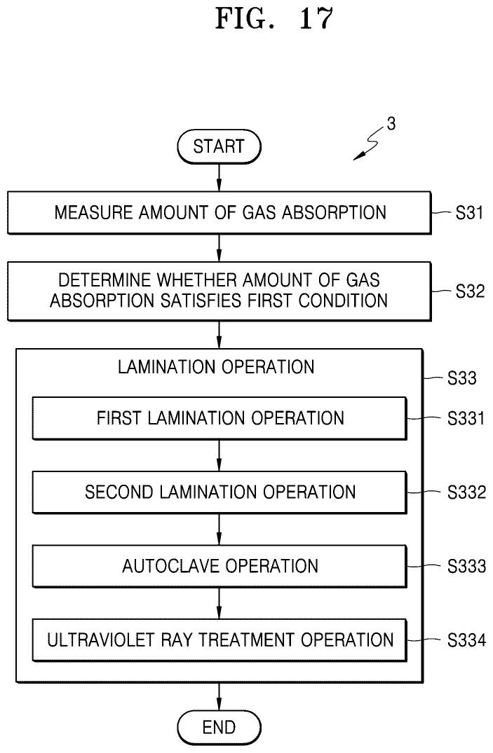

FIG. 17 is a schematic flowchart of a method for manufacturing a display device, according to an embodiment.

In FIG. 17, the same reference numerals as those in FIGS. 8 to 16 denote the same members, and thus repeated descriptions will be omitted.

Referring to FIGS. 8 to 17, a manufacturing method 3 of manufacturing a display device may include operation S31 of measuring a gas absorption amount, operation S32 of determining whether the gas absorption amount satisfies the first condition, and a lamination operation S33.

Referring to FIGS. 9 and 17, operation S31 of measuring an amount of gas absorption may be an operation in which the gas absorption amount measuring unit 21 measures an amount of gas absorption of the adhesive layer OCA at a first pressure and a first temperature.

In operation S32 of determining whether the gas absorption amount satisfies the first condition, when the gas absorption amount measurement unit 21 measures the amount of gas absorption of the adhesive layer OCA, the controller 23 may determine whether the measured gas absorption amount satisfies the first condition.

Referring to FIGS. 10 to 13 and 17, in the lamination operation S33, when the amount of gas absorption satisfies the first condition, the adhesive layer OCA and the cover window 700 may be laminated on the display panel 10P. The lamination operation S33 may include a first lamination operation (S331), a second lamination operation (S332), an autoclave process operation (S333), and an ultraviolet ray treatment operation (S334).

Referring to FIGS. 10 and 17, the first lamination operation (S331) may be an operation of laminating the adhesive layer OCA on the display panel 10P. Referring to FIGS. 11 and 17, the second lamination operation (S332) may be an operation of laminating the cover window 700 on the adhesive layer OCA. Referring to FIGS. 12 and 17, the autoclave process operation (S333) may be an operation of performing an autoclave process on the display device 1 at a second pressure and a second temperature. Referring to FIGS. 13 and 17, the ultraviolet ray treatment operation (S334) may be an operation of irradiating ultraviolet rays to the display device 1.

As such, the disclosure has been described with reference to an embodiment shown in the drawings, but this is merely an example, and those skilled in the art will understand that various modifications and variations of the embodiment are possible therefrom. Therefore, the technical scope of protection of the disclosure should be determined by the technical spirit of the appended claims.

According to embodiments of the disclosure, bubbles dissolved in the adhesive layer visible from the outside of the display device may be reduced.

The effects of the disclosure are not limited to the effects mentioned above, and other effects not mentioned will be clearly understood by those skilled in the art from the description of the claims.

It should be understood that embodiments described herein should be considered in a descriptive sense only and not for purposes of limitation. Descriptions of features or aspects within each embodiment should typically be considered as available for other similar features or aspects in other embodiments. While one or more embodiments have been described with reference to the figures, it will be understood by those of ordinary skill in the art that various changes in form and details may be made therein without departing from the spirit and scope as defined by the following claims.

What is claimed is:

1. A manufacturing apparatus for manufacturing a display device, the display device comprising a display panel, an adhesive layer, and a cover window, the manufacturing apparatus comprising:
a gas absorption amount measuring unit that measures an amount of gas absorption of the adhesive layer at a first pressure and a first temperature;
a lamination unit that laminates the adhesive layer and the cover window on the display panel; and
a controller that controls the lamination unit based on information measured by the gas absorption amount measurement unit,
wherein the controller controls the lamination unit to perform a lamination process only when the amount of gas absorption of the adhesive layer measured by the gas absorption amount measurement unit satisfies a first condition.

2. The manufacturing apparatus of claim 1, wherein the lamination unit comprises:
a first lamination unit that laminates the adhesive layer on the display panel;
a second lamination unit that laminates the cover window on the adhesive layer;
an autoclave unit that performs an autoclave process on the display device at a second pressure and a second temperature; and
an ultraviolet ray treatment unit that treats the display device with ultraviolet rays.

3. The manufacturing apparatus of claim 2, wherein the first temperature and the second temperature are equal to each other.

4. The manufacturing apparatus of claim 2, wherein the first pressure and the second pressure are equal to each other.

5. The manufacturing apparatus of claim 1, wherein the amount of gas absorption of the adhesive layer is an absorption amount of N₂ of the adhesive layer.

6. The manufacturing apparatus of claim 5, wherein the first pressure is 8 bar and the first temperature is 60 degrees Celsius.

7. The manufacturing apparatus of claim 6, wherein the first condition is less than or equal to about 0.07 mg/g.

8. The manufacturing apparatus of claim 1, wherein the adhesive layer comprises an optically transparent adhesive.

9. A method for manufacturing a display device, the display device comprising a display panel, an adhesive layer, and a cover window, the method comprising:
measuring an amount of gas absorption of the adhesive layer at a first pressure and a first temperature;
determining whether the amount of gas absorption of the adhesive layer satisfies a first condition; and
laminating the adhesive layer and the cover window on the display panel when the amount of gas absorption satisfies the first condition.

10. The method of claim 9, wherein the laminating comprises:
a first lamination operation of laminating the adhesive layer on the display panel;
a second lamination operation of laminating the cover window on the adhesive layer;
performing an autoclave process on the display device at a second pressure and a second temperature; and
treating the display device with ultraviolet rays.

11. The method of claim 10, wherein the first temperature and the second temperature are equal to each other.

12. The method of claim 10, wherein the first pressure and the second pressure are equal to each other.

13. The method of claim 9, wherein the amount of gas absorption of the adhesive layer is an absorption amount of N₂ of the adhesive layer.

14. The method of claim 13, wherein the first pressure is 8 bar and the first temperature is 60 degrees Celsius.

15. The method of claim 14, wherein the first condition is less than or equal to about 0.07 mg/g.

16. The method of claim 9, wherein the adhesive layer comprises an optically transparent adhesive.
